(19) United States
(12) Patent Application Publication
(10) Pub. No.: US 2009/0163840 A1
(43) Pub. Date: Jun. 25, 2009

(54) Massaging and aches relieving pad for a support

(76) Inventor: Pang-Ching Chiang, Taipei (TW)

Correspondence Address: Joe McKinney Muncy, PO Box 1364, Fairfax, VA, 22038-1364, US

(21) Appl. No.: 12/000,986

(22) Filed: Dec. 19, 2007

(51) Int. Cl. A61F 5/00

(52) U.S. Cl. 602/5

(57) ABSTRACT

A massaging and aches relieving pad for a support has a main body with an inner surface and multiple elliptical protrusions. The elliptical protrusions are formed concentrically on and protrude respectively from the inner surface of the main body and linearly abut joints. Therefore, the pad not only prevents impacts or injury of the joints but also massages and relieves aches in the joints.

### BACKGROUND OF THE INVENTION

1. Field of the Invention

The present invention relates to a pad, especially to a pad for a support that has multiple concentric ellipses for massaging joints and relieving aches in the joints.

2. Description of the Prior Arts

A support is worn around joints including, but not limited to the knee, ankle, wrist and the like and has a pad being mounted in the support and corresponding to the joints. The pad in the support may appropriately restrict the movement of the joints to prevent impact and undesired movement. Therefore, the support protects well joints from being injured, aides in recover of injured joints and prevents previously injured joints from being injured again.

A conventional pad being mounted in the support has an inner surface and multiple protrusions. The inner surface corresponds to a corresponding joint when the support is worn around the joint. The protrusions are bulges separately formed on the inner surface of the conventional pad and abutting the joint to protect and massage the joint and relieve aches in the joint. Although the protrusions massage the joints, their shape constantly pokes the joint causing discomfort.

To overcome the shortcomings, the present invention provides a massaging and aches relieving pad for a support to mitigate or obviate the aforementioned problems.

### SUMMARY OF THE INVENTION

The main objective of the present invention is to provide a massaging and aches relieving pad for a support that has a main body with an inner surface and multiple elliptical protrusions. The elliptical protrusions are formed concentrically on and protrude respectively from the inner surface of the main body and linearly abut joints. Therefore, the pad not only prevents impacts or injury of the joints but also massages and relieves aches in the joints.

Other objectives, advantages and novel features of the invention will become more apparent from the following detailed description when taken in conjunction with the accompanying drawings.

### BRIEF DESCRIPTION OF THE DRAWINGS

FIG. 1 is a perspective view of a first embodiment of a massaging and aches relieving pad for a support in accordance with the present invention;

FIG. 2 is a perspective view of a second embodiment of a massaging and aches relieving pad for a support in accordance with the present invention;

FIG. 3 is a cross-sectional side view of a first embodiment of the pad in FIG. 2;

FIG. 4 is a cross-sectional side view of a second embodiment of the pad in FIG. 2;

FIG. 5 is an operational perspective view of the pad in FIG. 2 being mounted in the support and being shown in phantom lines; and

FIG. 6 is an operational perspective view of the pad in FIG. 2 being mounted in the support, abutting an ankle and being shown in phantom lines.

### DETAILED DESCRIPTION OF THE PREFERRED EMBODIMENTS

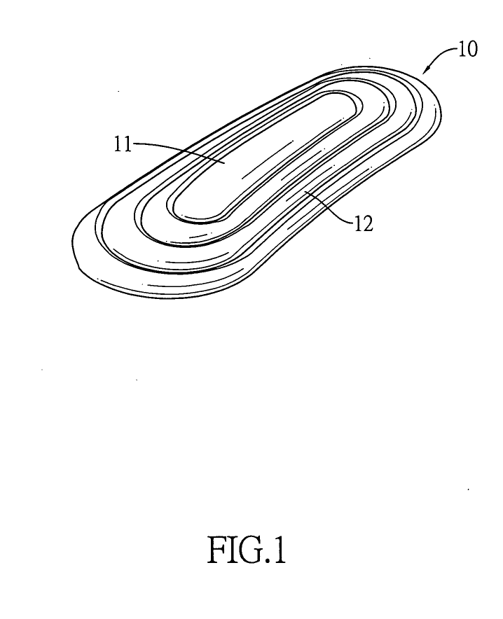

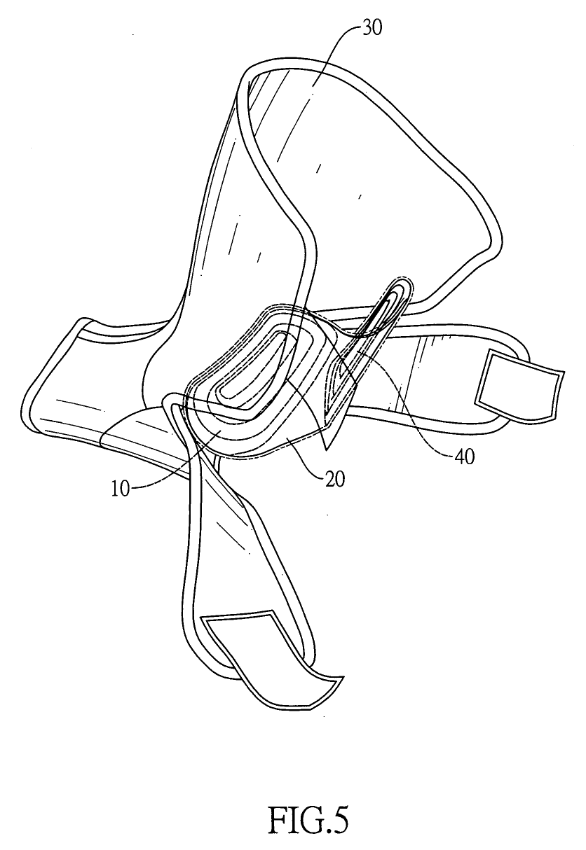

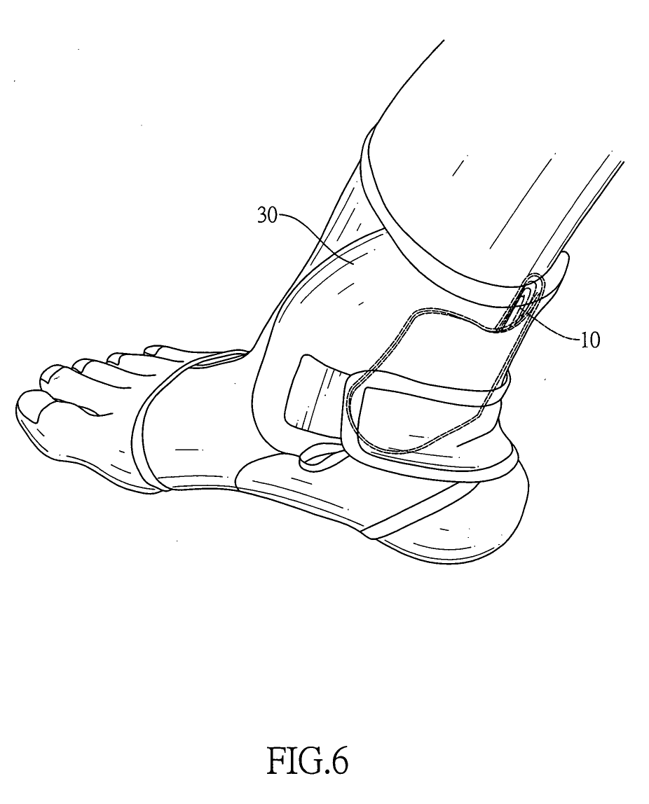

With reference to FIGS. 1, 5 and 6, a massaging and aches relieving pad for a support in accordance with the present invention may be mounted in a support (30) for ankles, elbows, knees or the like, may be resilient and soft, may be silicone, plastic foam or the like and comprises a main body (10, 10′), at least one optional additional body (40, 40′) and at least one optional connector (20).

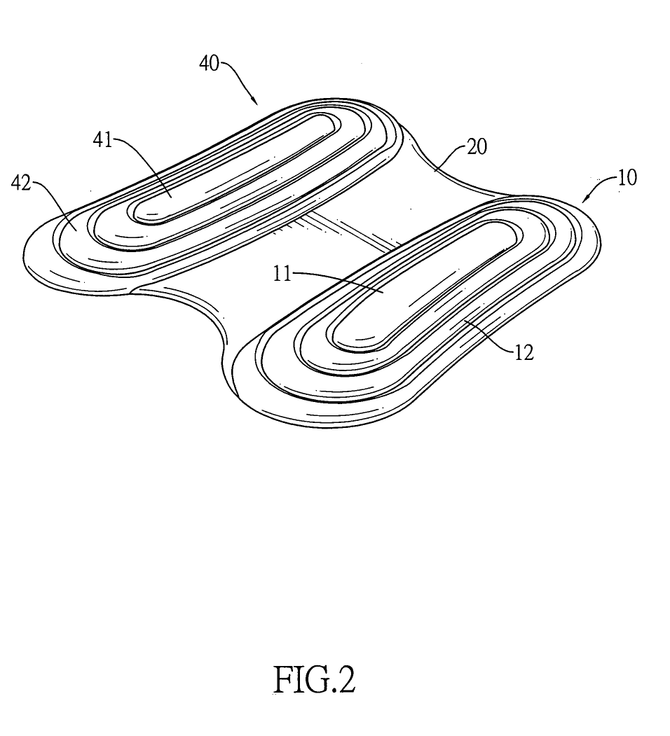

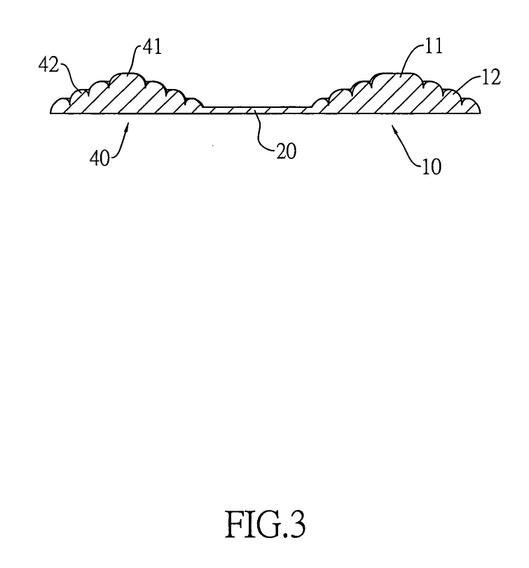

With further reference to FIGS. 2 and 3, each body (10, 10′, 40, 40′) has an inner surface, an edge, an optional central protrusion (11, 11′, 41, 41′) and multiple elliptical protrusions (12, 12′, 42, 42′).

The central protrusion (11, 11′, 41, 41′) is formed on and protrudes from the inner surface of the body (10, 10′, 40, 40′) and has a protruding distance.

The elliptical protrusions (12, 12′, 42, 42′) are formed concentrically on and respectively protrude from the inner surface of the body (10, 10′, 40, 40′), are formed concentrically around the central protrusion (11, 11′, 41, 41′) of the body (10, 10′, 40, 40′) and each elliptical protrusion (12, 12′, 42, 42′) has a protruding distance.

With reference to FIG. 3, the protruding distances of the elliptical protrusions (12, 42) may be shorter than the protruding distance of the central protrusion (11, 41) and increase closer toward the central protrusion (11, 41).

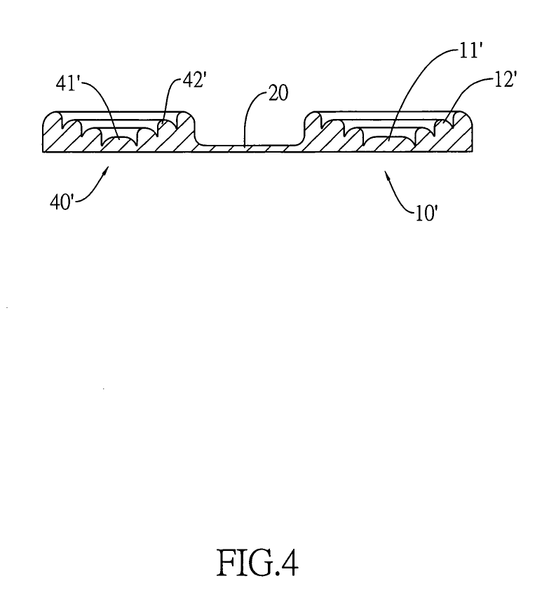

With reference to FIG. 4, the protruding distances of the elliptical protrusions (12′, 42′) may be longer than the protruding distance of the central protrusion (11′, 41′) and decrease closer to the central protrusion (11′, 41′).

Each connector (20) is a same material as the bodies (10, 10′, 40, 40′) and respectively connects between two adjacent edges of the adjacent bodies (10, 10′, 40, 40′).

The massaging and aches relieving pad for a support as described has the following advantages. The central and elliptical protrusions (11, 11′, 12, 12′, 41, 41′, 42, 42′) of the bodies (10, 10′, 40, 40′) linearly abut the joints. Therefore, the pad as described not only prevents impacts or injury to the joints but also massages and relieves aches in the joints.

Even though numerous characteristics and advantages of the present invention have been set forth in the foregoing description, together with details of the structure and features of the invention, the disclosure is illustrative only. Changes may be made in the details, especially in matters of shape, size, and arrangement of parts within the principles of the invention to the full extent indicated by the broad general meaning of the terms in which the appended claims are expressed.

What is claimed is:

1. A massaging and aches relieving pad for a support comprising a main body having
an inner surface;
an edge; and
multiple elliptical protrusions being formed concentrically on and respectively protruding from the inner surface of the main body and each elliptical protrusion having a protruding distance.

2. The massaging and aches relieving pad for a support as claimed in claim 1, wherein the pad further comprises
at least one additional body, each one of the at least one additional body having
an inner surface;
an edge; and
multiple elliptical protrusions being formed concentrically on and respectively protruding from the inner surface of the additional body and each elliptical protrusion having a protruding distance; and
at least one connector respectively connecting between two adjacent edges of adjacent bodies.

3. The massaging and aches relieving pad for a support as claimed in claim 1, wherein
the main body further has a central protrusion being formed on and protruding from the inner surface of the main body and having a protruding distance; and
the elliptical protrusions of the main body are formed concentrically around the central protrusion of the main body.

4. The massaging and aches relieving pad for a support as claimed in claim 2, wherein
each body has a central protrusion being formed on and protruding from the inner surface of the body and having a protruding distance; and
the elliptical protrusions of each body are formed concentrically around the central protrusion of the body.

5. The massaging and aches relieving pad for a support as claimed in claim 3, wherein the protruding distances of the elliptical protrusions of the main body are shorter than the protruding distance of the central protrusion and increase toward the central protrusion.

6. The massaging and aches relieving pad for a support as claimed in claim 3, wherein the protruding distances of the elliptical protrusions of the main body are longer than the protruding distance of the central protrusion and reduce toward the central protrusion.

7. The massaging and aches relieving pad for a support as claimed in claim 4, wherein the protruding distances of the elliptical protrusions of each body are shorter than the protruding distance of the central protrusion of the body and increase toward the central protrusion.

8. The massaging and aches relieving pad for a support as claimed in claim 4, wherein the protruding distances of the elliptical protrusions of each body are longer than the protruding distance of the central protrusion of the body and reduce toward the central protrusion.

9. The massaging and aches relieving pad for a support as claimed in claim 5, wherein the pad is resilient and soft.

10. The massaging and aches relieving pad for a support as claimed in claim 6, wherein the pad is resilient and soft.

11. The massaging and aches relieving pad for a support as claimed in claim 7, wherein the pad is resilient and soft.

12. The massaging and aches relieving pad for a support as claimed in claim 8, wherein the pad is resilient and soft.

13. The massaging and aches relieving pad for a support as claimed in claim 9, wherein the pad is silicone.

14. The massaging and aches relieving pad for a support as claimed in claim 10, wherein the pad is silicone.

15. The massaging and aches relieving pad for a support as claimed in claim 11, wherein the pad is silicone.

16. The massaging and aches relieving pad for a support as claimed in claim 12, wherein the pad is silicone.

17. The massaging and aches relieving pad for a support as claimed in claim 9, wherein the pad is plastic foam.

18. The massaging and aches relieving pad for a support as claimed in claim 10, wherein the pad is plastic foam.

19. The massaging and aches relieving pad for a support as claimed in claim 11, wherein the pad is plastic foam.

20. The massaging and aches relieving pad for a support as claimed in claim 12, wherein the pad is plastic foam.
